(19) United States
(12) Patent Application Publication
(10) Pub. No.: US 2022/0184410 A1
(43) Pub. Date: Jun. 16, 2022

(54) Method and System of Light Therapy For Disaggregation of Red Blood Cells and Treatment of Respiratory Illness

(71) Applicant: Advanced Light Devices, LLC, Las Vegas, NV (US)

(72) Inventors: Eugene Barnett, Las Vegas, NV (US); James Smeriglio, Jupiter, FL (US)

(21) Appl. No.: 17/551,074

(22) Filed: Dec. 14, 2021

Related U.S. Application Data
(60) Provisional application No. 63/125,432, filed on Dec. 15, 2020.

(51) Int. Cl. A61N 5/06

(52) U.S. Cl. CPC A61N 5/062; A61N 2005/0645; A61N 2005/0663

(57) ABSTRACT

A prosthetic liner for use with a prosthetic socket that has a thermoplastic elastomeric (TPE) layer that is in contact with the amputee's residual limb. The TPE layer is not uniform in thickness but has ridges to extend circumferentially around the TPE layer. A fabric exterior layer covers the outer surface of the TPE layer conforming to the ridges and is used as a substrate to form a mechanical bond to an elastomeric material. Impregnating the elastomeric material within the fabric exterior occurs by applying an uncured material to the fabric exterior layer and onto at least the apex surfaces located within the sealing regions thereby creating an air tight boundary layer when inserted into a socket. When a vacuum is applied the air is evacuated from the volume below the seal layer.

### CROSS-REFERENCE TO RELATED APPLICATIONS

This application claims priority to U.S. Provisional Application No. 62/125,432, filed Dec. 15, 2020, the contents of which are hereby incorporated by reference herein.

### BACKGROUND OF THE INVENTION

### Field of the Invention

This invention relates to a method and system of light therapy for disaggregation of red blood cells and treatment of certain diseases. More particularly, this invention relates to a method of treatment for pulmonary hypertension using a self-powered wearable light emitting device which emits light at certain wavelengths over a certain frequency thereby aiding in the disaggregation of red blood cells.

### Description of the Background Art

Pulmonary hypertension is a condition of increased blood pressure within the arteries of the lungs. Symptoms include shortness of breath, syncope, tiredness, chest pain, swelling of the legs, and a fast heartbeat. This condition may also make it difficult to exercise. Onset is typically gradual.

The cause of pulmonary hypertension is often unknown. Risk factors include a family history, prior blood clots in the lungs, HIV/AIDS, sickle cell disease, cocaine use, chronic obstructive pulmonary disease, sleep apnea, living at high altitudes, and problems with the mitral valve. The underlying mechanism typically involves inflammation and subsequent remodeling of the arteries in the lungs. Diagnosis involves first ruling out other potential issues.

There is currently no known cure for pulmonary hypertension, although research for a cure is ongoing. Treatment depends on the type of disease. A number of supportive measures such as oxygen therapy, diuretics, and medications to inhibit blood clotting may be used. Medications specifically used to treat pulmonary hypertension include epoprostenol, treprostinil, iloprost, bosentan, ambrisentan, macitentan, and sildenafil. Lung transplantation may be an option in severe cases.

While the exact frequency of the condition is unknown, it is estimated that about 1,000 new cases occur a year in the United States. Females are often more affected than males. Onset is typically between 20 and 60 years of age. There are a number of supportive measures besides medicinal treatment such as oxygen therapy and diuretics. Each of the medicinal treatments used to treat pulmonary hypertension have several side effects and drug interactions. Endothelin receptor antagonists have been associated with birth defects and liver damage. A patient's doctor may request monthly liver function tests to prevent irreversible liver damage. Likewise, PDE-5 inhibitors should not be taken in combination with nitrates, nitric oxide donors, or alpha blockers, as taking these drugs in combination may cause a significant drop in blood pressure, which could result in loss of consciousness or even death. Prostacyclins tell you blood vessels to dilate which becomes dangerous when mixed with other medications that have the same function such as diuretics, antihypertensives, and other vasodilators because they may lower blood pressure too much. Warfarin may also disadvantageously interact with other medicines to dangerously lower blood pressure and can cause bruising and other signs of internal bleeding.

In addition, methylene blue has been found to be a drug that is helpful in the prevention and treatment of certain respiratory diseases. Methylene blue is a tricyclic phenothiazine compound approved for the treated of acquired methemoglobinemia and other uses. It has been found, by virtue of the present invention, that methylene blue, when used in combination with light at certain wavelengths, contributes to the prevention and treatment of severe acute respiratory syndrome-coronavirus 2 (SAR-CoV-2), otherwise known as COVID-19.

Therefore, it is an object of this invention to provide an improvement which overcomes the aforementioned inadequacies of the prior art devices and provides an improvement which is a significant contribution to the advancement of red blood cell disaggregation art.

Another objective of the invention is to provide a method of treating pulmonary hypertension without the use of drugs or medicinal treatment.

Another objective of the invention is to provide a system which will photo-activate light sensitive molecules in the human body.

Another objective of the invention is to provide a method of treating respiratory diseases using light therapy.

Another objective of the invention is to provide a system for the treatment of the aforementioned maladies via a wearable device.

Another objective of the invention is to provide pulsed light therapy so as to avoid potential burning of the user's skin.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a method of treatment for respiratory illnesses using light therapy. Specifically, the present invention relates to a method of treatment comprising alternating LED lights having wavelengths between approximately 625 nanometers and 670 nanometers; alternating between the two wavelengths at a frequency of 432 milliseconds; pulsing the active LEDs for 60 nanoseconds on and 40 nanoseconds off; and turning off the LED lights after treatment is finished. The present invention is part of a system that delivers the method via a device that is wearable on a user's wrist.

The timing of the LED lights flickering is important to prevent overheating and burning of the user's skin. In addition, the wavelengths used are specially determined to be ideal for the disaggregation of red blood cells because they are in the red spectrum of the visible light range.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a side perspective view of the light therapy device that forms part of the system of the present invention;

FIG. 2 is a bottom perspective view of the light therapy device;

FIG. 3 is a top view of the light therapy device;

FIG. 4 is a bottom view of the light therapy device;

FIG. 5 is a left side view of the light therapy device;

FIG. 6 is an right side view of the light therapy device;

FIG. 7 is a front side view of the light therapy device;

FIG. 8 is a back side view of the light therapy device; and

FIG. 9 is a perspective view of the light therapy device attached to an unclaimed wristband; and

FIG. 10 is a flow chart of the preferred method of the present invention.

Similar reference numerals refer to similar parts throughout the several views of the drawings.

### DETAILED DESCRIPTION OF THE PREFERRED EMBODIMENTS

The following description is of the best mode presently contemplated for carrying out the invention. This description is not to be taken in a limiting sense, but is made merely for the purpose of describing one or more preferred embodiments of the invention. The scope of the invention should be determined with reference to the claims.

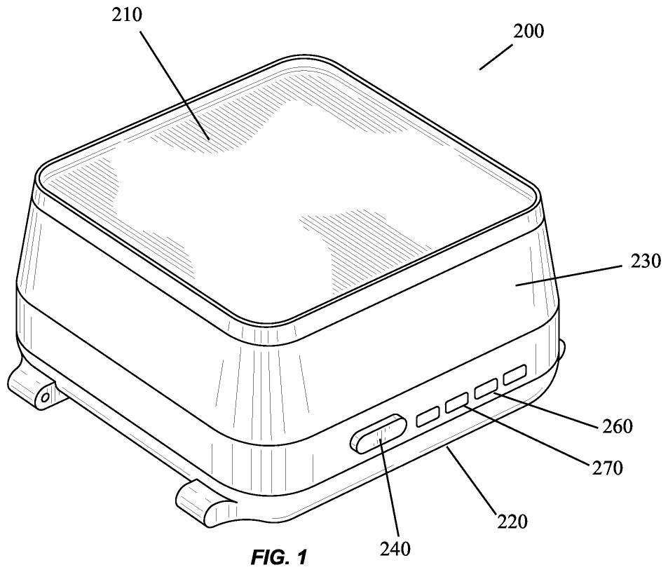

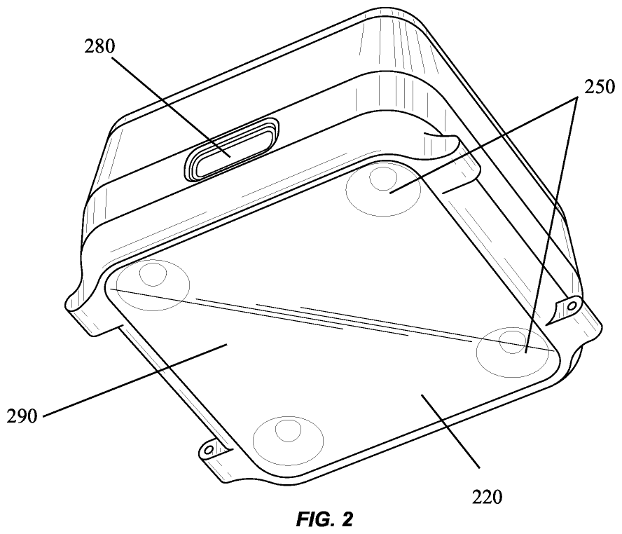

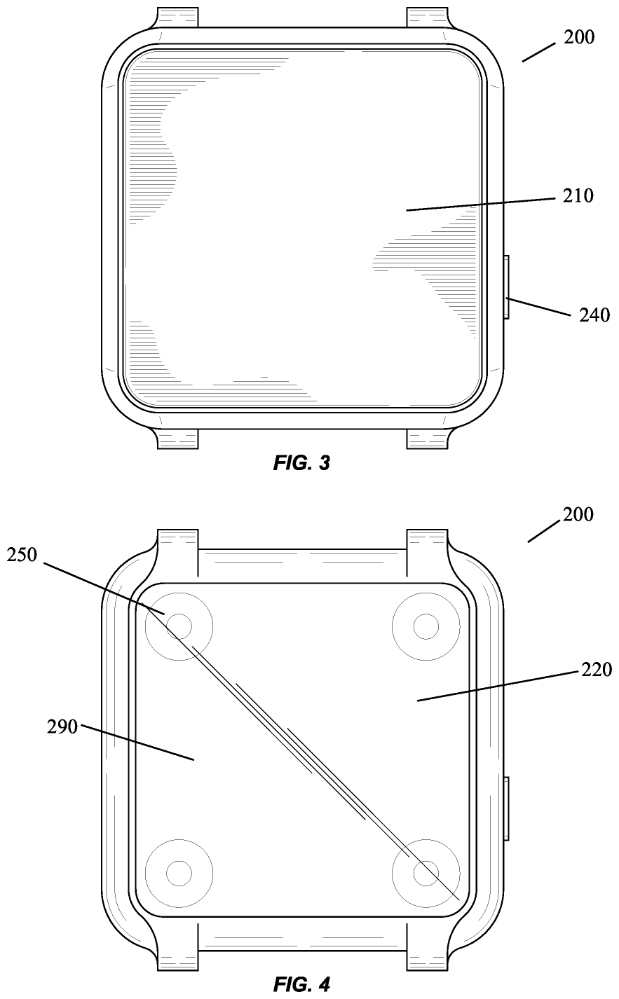

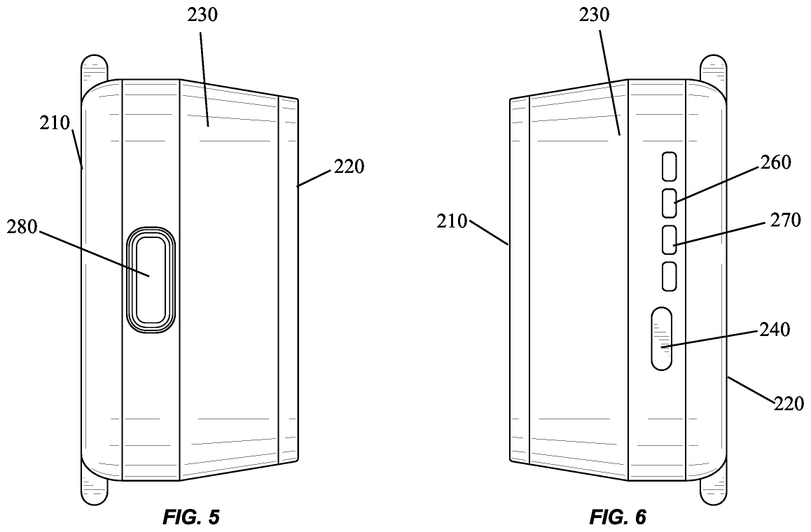

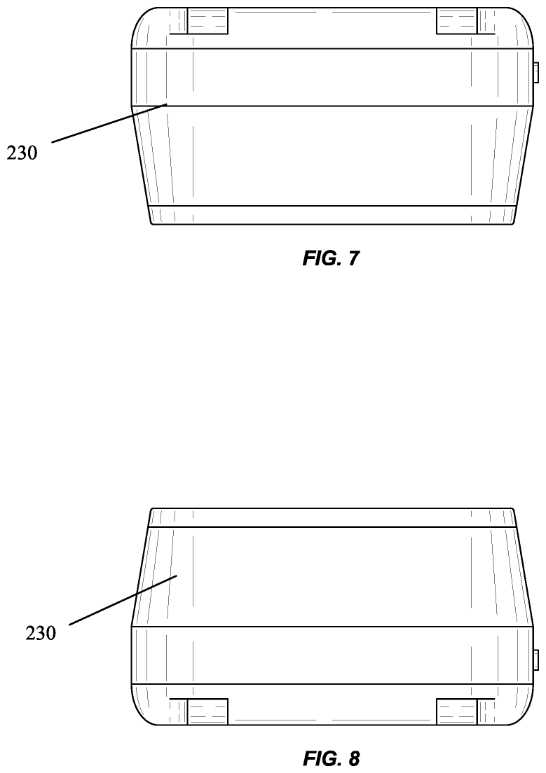

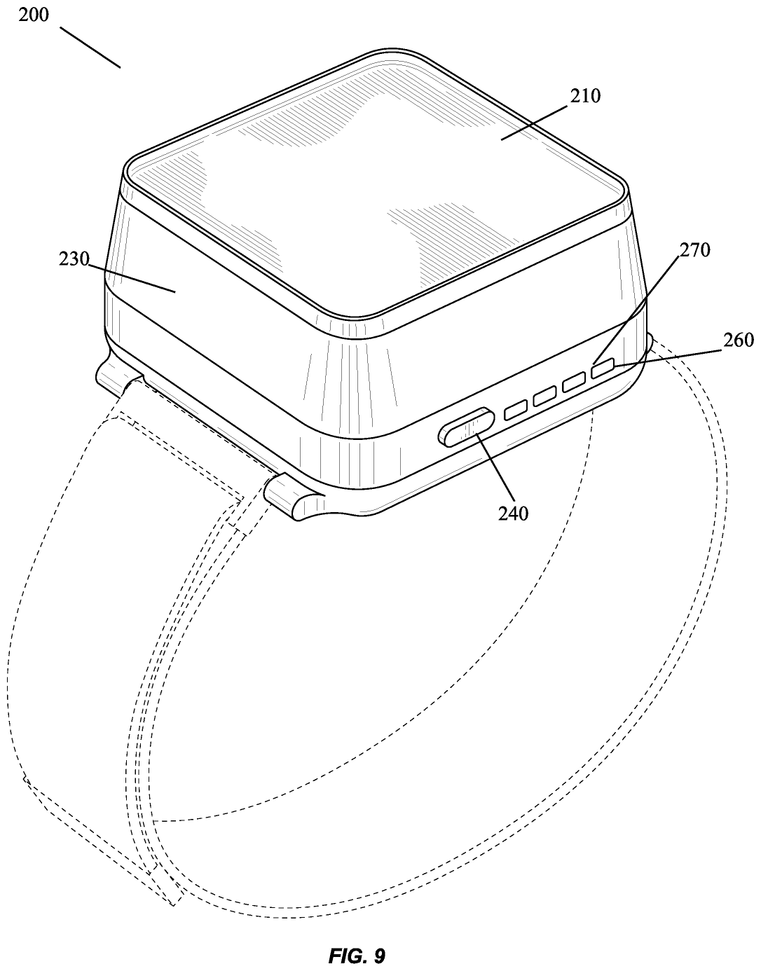

As shown in FIGS. 1-9, a wearable device 200 is the preferred way of delivering the method described above. This device 200 has a top face 210 and a bottom face 220 as well as side walls 230. Preferably, the device 200 has a debounce button 240 that allows the user to turn the device 200 on and off to start and stop the therapy method of the present invention. The bottom face 220 has a plurality of light sources 250 embedded within underneath a clear face 290 in that faces the user's skin on the desired appendage. The device is preferably attached to a user's wrist but can be attached to any appendage that has sufficient access to a user's arteries. The side walls 230 also include a battery indicator 260 which preferably has a plurality of indication lights 270 to inform the user of the battery level. The device may also include a USB-C charging port 280 in the side walls 230.

This light therapy can take place whether or not a user has received methylene blue. The benefit to the user receiving methylene blue and then partaking in the method described above is that methylene blue when bombarded with light at the wavelengths described herein allows for the creation of free radicals via reactive oxygen species within the user's system. These free radicals can then interact with viruses, bacteria, and other microorganisms that negatively affect the health of a user. The use of methylene blue in combination with the light therapy described herein has also been shown to be an effective preventative treatment of COVID-19.

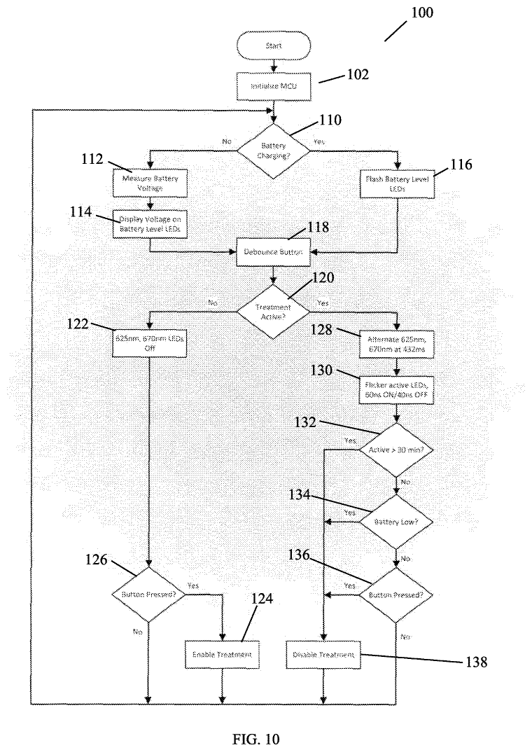

FIG. 10 illustrates a flow chart of the method 100 of the present invention. The first step after powering up the device 102, involves answering the question of whether the battery is charging 110. If not, the battery voltage is measured 112 and the voltage is displayed 114. If the battery is charging, then LEDs flash showing the battery level 116. When treatment is desired, a debounce button 240, as shown in FIGS. 1-9, is pressed 118. If treatment is not active when the debounce button is pressed 120, a plurality of light sources 250, preferably light emitting diodes (LEDs), are off 122 which enables treatment 124 when the debounce button is pressed 126. If treatment is active 120, then the plurality of light sources 250 begin alternating at wavelengths of approximately 625 nanometers and approximately 670 nanometers at a frequency of approximately 432 milliseconds 128. The plurality of light sources are pulsed approximately 60 nanoseconds on and approximately 40 nanoseconds off 130 to avoid burning the user's skin. The use of these wavelengths for the period of time disclosed herein allows for deep penetration into the arteries of the body in order to infuse energy into the blood and to photoactivate photosensitive molecules such as red blood cells. This is a topical method of delivering intense photo-red wavelengths into the blood for the disaggregation of red blood cells. The therapy then has a maximum therapy time of about 30 minutes at which point the therapy ends 132. Likewise, if the battery on the device delivering the light energy is low then the therapy will end 134. Finally, if the user desires to end the therapy early, then they can press the debounce button and end the therapy 136. Otherwise, the therapy will continue 138.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A method of treating respiratory illnesses by disaggregating red blood cells comprising:
exposing a user to a plurality of light sources having a wavelength;
alternating the wavelength of the light sources between approximately 625 nanometers and approximately 670 nanometers at a frequency of approximately 432 milliseconds;
pulsing the plurality of light sources for approximately 60 nanoseconds on and approximately 40 nanoseconds off; and
powering down the plurality of light sources after an active therapy session of no more than 30 minutes.

2. The method of claim 1 wherein the user intakes a medically effective amount of methylene blue prior to being exposed to the plurality of light sources.

3. The method of claim 1 wherein the light sources are light emitting diodes.

4. A system for the treatment of respiratory illnesses comprising:
a user's appendage;
a wearable device comprising a top face, a bottom face, and side walls, wherein the bottom face is embedded with a plurality of light sources and wherein the side walls includes at least one battery indicator, strapped to the user's appendage such that the bottom face is in contact with the user's appendage and wherein the wearable device applies a light therapy, the light therapy further comprising:
exposing the user's appendage to the plurality of light sources wherein each light source has a wavelength;
alternating the wavelength of the light sources between approximately 625 nanometers and approximately 670 nanometers at a frequency of approximately 432 milliseconds;
pulsing the plurality of light sources for 60 nanoseconds on and 40 nanoseconds off; and
powering down the plurality of light sources after an active therapy session of no more than 30 minutes.

5. The system of claim 4 wherein the user intakes a medically effective amount of methylene blue prior to be being exposed to the plurality of light sources.

6. The system of claim 4 wherein the light sources are light emitting diodes.
